(19) United States
(12) Patent Application Publication
(10) Pub. No.: US 2006/0224413 A1
(43) Pub. Date: Oct. 5, 2006

(54) Method and system for providing tele-healthcare by using household medical devices

(75) Inventors: Minjoon Kim, Daejeon (KR); Junggon Kim, Seoul (KR); Miseon Yoon, Daejeon (KR)

Correspondence Address: LAHIVE & COCKFIELD, 28 STATE STREET, BOSTON, MA, 02109, US

(73) Assignee: H3 SYSTEM CO., LTD, Yuseong-gu (KR)

(21) Appl. No.: 10/552,935

(22) PCT Filed: Apr. 8, 2004
(86) PCT No.: PCT/KR04/00822

§ 371 Date: Oct. 11, 2005

(30) Foreign Application Priority Data
Apr. 9, 2003 (KR) 1020030022365

(51) Int. Cl. A61B 5/00

(52) U.S. Cl. 705/2; 600/301

(57) ABSTRACT

Disclosed is an on-line healthcare system using a domestic medical device and a method thereof. The on-line healthcare system comprises a portable measurement unit for performing a biological measurement for diagnosing a user's health and converting measured data so as to generate biological measurement information data and/or measurement information data including a part of the biological measurement data; and a cradle connected to the portable measurement unit so as to automatically transmit/receive the measurement information data to/from the portable measurement unit by means of a program accommodated therein. The on-line healthcare system is economical because the on-line healthcare system does not require an additional personal computer in order to provide tele-healthcare services. Also, the on-line healthcare system does not require additional handling in order to transfer data, so that usage of the on-line healthcare system is simple.

### TECHNICAL FIELD

The present invention relates to a method and a system for providing tele-healthcare by using household medical devices, and more particularly to a method and a system for providing tele-healthcare, in which if a user couples a portable measurement unit with a cradle after measuring biological information on user's health by using the portable measurement unit at a home or office, biological measurement data are transmitted to the cradle, which is automatically connected with a server linked to a communication network upon receiving the biological measurement data, so that measurement information data including the biological measurement data are transmitted to the server for allowing the user to receive an opinion of a medical specialist based on the measurement information data.

### BACKGROUND ART

In general, a patient should in person go to a hospital in order to take medical treatment from a doctor and may have to take a medicine prepared from a pharmacy according to prescription of the doctor. Rarely, a doctor goes out to see a patient in order to treat the patient. However, since the patient must bear much medical expense and the doctor incurs a great time loss, such a case rarely happens.

As described above, a so-called “direct medical treatment system” allowing a patient to directly take medical treatment from a doctor incurs many inconveniences in that the patient must in person go to a hospital in order to receive medical care. Even though the patient visits a hospital, the patient must wait for seeing a doctor for a long time. In particular, in rural areas having no general hospital, since a patient cannot receive medical treatment from a medical specialist, the patient is treated by a general practitioner in most cases. For this reason, it is difficult to exactly diagnose a disease, so that a patient having an disease cannot take the treatment at an early stage of the disease in most cases.

In order to solve the above problem, medical consulting and treatment have been provided through an on-line system assisted by the development of the Internet. Currently, an on-line medical consulting is legally permitted, but on-line medical treatment or on-line prescription is restricted.

However, a currently suggested medical bill intends to give effect to an electronic medical record (EMR) (e.g., electronic prescription). A new medical amendment bill includes contents of permitting a medical act through communication media if there are considerable reasons to match with benefit of a patient. If an electronic prescription, which is obtained by a medical act through communication media, is legally permitted, a user can take medicine from a pharmacy according to an electronic prescription. Furthermore, a user can take a general medicine except for a special medicine from a pharmacy called an “Internet pharmacy”.

However, even though on-line medical treatment is not legally permitted, an encouraged by the fact that information network has been widely deployed, people are interested in home treatment or tele-treatment, so that healthcare sites or virtual hospitals have been open. This is because an on-line medical advice can offer convenience to both patients and doctors. However, currently provided tele-healthcare systems have a problem in that usage thereof is inconvenient and a cost thereof is very expensive. Accordingly, the currently provided tele-healthcare systems are not widely used. In order to improve such conventional tele-healthcare systems, a system allowing a patient to examine health conditions of the patient at home and to take medical advice based on health information data with a low cost, has been discussed.

In general, when a patient goes to a hospital, the patent must take basic examinations for diagnosis of diseases such as a pulse examination, a blood sugar examination, a body fat examination, and a urine examination. Since the basic examinations are used as an important clinical index in order to diagnose diseases and functions of various human organs and make a treatment plan, the basic examinations have been regarded as indispensable routine examinations. However, such basic examinations can be sufficiently performed at home by the user using only a suitable device while the user need not visit a hospital. Also, if the device is portable, the user can take examinations by using the device regardless of places. For these reasons, a plurality of devices capable of measuring such basic examinations at home has been suggested.

However, conventional devices are inconvenient in most cases. In addition, it is difficult for the old, the weak, and a patient, which may frequently use health measurement units, to use the conventional devices. Therefore, the health measurement units with simpler way of use have been demanded. Also, measurement units capable of providing communications are very expensive, so that it is difficult for an individual user to buy such measurement units. Therefore, more economical systems have been necessary in order for tele-diagnosis to be more popular.

FIG. 1 is a block diagram showing a structure of a conventional healthcare system.

As shown in FIG. 1, according to the conventional healthcare system, if a user 100 measures data by using a fixed measuring machine 102 and transfers measured data to the server 108 through the Internet 104, a LAN (not shown), or a wired telephone line (106), the server 108 analyzes the measured data so as to provide related services. The measuring machine 102 used for the related services is a multiple measuring machine, which is used for measuring blood pressure, blood sugar, and body fat. Herein, since the measuring machine 102 is not portable, the measuring machine 102 has a restriction of moving a place in measuring data. Also, since the measuring machine 102 is generally a multiple measuring machine, the measuring machine 102 is very expensive. In addition, since the measuring machine 102 performs multiple functions, the way of use for the measuring machine 102 is very complex and cumbersome.

FIG. 2 is a block diagram showing a structure of another conventional healthcare system.

The healthcare system shown in FIG. 2 is designed in order to solve the above problems of the system shown in FIG. 1. The healthcare system is a system for connecting a portable measuring unit 202 to a personal computer 204 so as to connect the personal computer with a server 208 though a modem, a LAN card, etc., for transmitting biological data to the server 208, and for receiving medical diagnosis results or medical tips from a medical specialist. Herein, the personal computer 204 stores healthcare software. The healthcare software displays an inspection result and a clinical diagnosis corresponding to the inspection result on LCD and outputs the inspection result for a human body and the clinical diagnosis for the inspection result through a printer. Also, the healthcare software continuously monitors inspection results accumulated for a month or for a year by storing the inspection results so as to provide each user with a disease symptom, health information, and a clinical diagnosis for the inspection result.

Generally, in order to make efficient use of the healthcare system shown in FIG. 2, a user must be good at handling the healthcare software. However, since such a kind of the healthcare software has a complex usage, it is not easy for the old and the weak or the patient to handle the healthcare software. Also, users must have a personal computer 204 connected with the Internet in order to use the healthcare system shown in FIG. 2. Therefore, users not having a personal computer 204 must bear additional expense in order to buy a personal computer.

Due to the above-described problems, in spite of the current trend described above, it is difficult to provide a system allowing a user to diagnose a disease and manage a diagnosis result at home. Also, although an available system exists, since the available system is very expensive, and therefore, cannot be widely or readily used. Also, it is difficult for ordinary users not having expert knowledge to interpret various health examination results. Disclosure of the Invention Therefore, the present invention has been made in view of the above-mentioned problems, and it is a first object of the present invention to provide a method and a system for providing healthcare, in which if a user couples a portable measurement unit with a cradle after frequently measuring biological information about his or her healthcare by using the portable measurement unit at his or her home or office without visiting a hospital or an office of a medical practitioner, the cradle is automatically connected with a server linked with an information network such as the Internet, PSTN, etc., through a modem, TCP/IP, etc., so as to transmit measurement information data to the server and so as to receive an opinion of a medical specialist about the measurement information data.

A second object of the present invention is to provide an on-line healthcare system and an on-line healthcare method, in which provide a tele-healthcare service capable of systematically managing current healthcare state of a user as well as disease transition by storing measurement information data such as biological measurement data, measurement time, etc., and opinion data of a doctor in a database of a server and continuously monitoring healthcare state of the user and employ a economical domestic medical device having a convenient usage.

According to an aspect of the present invention, there is provided an on-line healthcare system by using a domestic medical device, the on-line healthcare system comprising: a portable measurement unit for performing a biological measurement for diagnosing a user's health and converting measured data so as to generate biological measurement information data and/or measurement information data including the biological measurement data; and a cradle connected to the portable measurement unit so as to automatically transmit/receive the measurement information data to/from the portable measurement unit by means of a program stored therein.

According to another aspect of the present invention, there is provided an on-line healthcare method by using a domestic medical device including a portable measurement unit having a measurement part, a signal processing part, and a first communication module of the portable measurement unit, and a cradle having a program included therein and a second communication port of the cradle, the on-line healthcare method comprising the steps of: (a) allowing the cradle to perform biological measurement for diagnosing health of a user; (b) allowing the signal processing module to convert a result of the biological measurement into biological measurement data; (c) determining whether or not an emergency occurs according to an analysis result of the biological measurement data measured by the portable measurement unit; (d) transferring the measurement information data including a part of the biological measurement data to the cradle by using the second communication module of the cradle, the first communication module of the portable measurement unit, and the program included in the cradle, the cradle being automatically operated when the portable measuring unit makes contact with the cradle, if step (c) determines that no emergency occurs; and (e) transferring the measurement information data received by the cradle to the server by using the program included in the cradle and the second communication module of the cradle.

According to still another aspect of the present invention, there is provided an on-line healthcare method by using a domestic medical device including a portable measurement unit having a measurement part, a signal processing part and a first communication module of the portable measurement unit, and a cradle having a program included therein and a second communication port of the cradle, the on-line healthcare method comprising the steps of: (a) allowing the cradle to perform biological measurement for diagnosing health of a user; (b) allowing the signal processing module to convert a result of the biological measurement into biological measurement data; (c) transferring the measurement information data including a portion of the biological measurement data to the cradle by using the first communication module of the portable measurement unit, the second communication module of the cradle, and the program included in the cradle, the cradle being automatically operated when the portable measuring unit is contacted with the cradle; and (d) transferring the measurement information data received by the cradle to the server by using the program included in the cradle and the second communication module of the cradle.

### BRIEF DESCRIPTION OF THE DRAWINGS

The foregoing and other objects, features and advantages of the present invention will become more apparent from the following detailed description when taken in conjunction with the accompanying drawings in which:

FIG. 1 is a block diagram showing a structure of a conventional healthcare system;

FIG. 2 is a block diagram showing a structure of another conventional healthcare system;

FIG. 3 is a schematic view showing a structure of a healthcare system according to a preferred embodiment of the present invention;

FIG. 4 is a schematic view showing internal structures of a portable measurement unit and a cradle and a method of coupling the portable measurement unit with the cradle according to a preferred embodiment of the present invention;

FIG. 5 is a view showing a structure for coupling a portable measurement unit with a cradle through communication ports according to a preferred embodiment of the present invention;

FIG. 6 is a view showing a structure of coupling the portable measurement unit with a cradle by means of concave and convex electrodes according to a preferred embodiment of the present invention;

FIG. 7 is a view showing a structure in which a first communication port of a portable measurement unit and a second communication port of a cradle are exposed only when they are coupled with each other according to a preferred embodiment of the present invention;

FIG. 8 is a view showing a structure in which a first communication port of a portable measurement unit and a second communication port of a cradle are coupled with each other without electric contact according to a preferred embodiment of the present invention;

FIG. 9 is a flow chart showing an operation of a portable measurement unit according to a preferred embodiment of the present invention;

FIG. 10 is a flow chart representing a process in which a cradle downloads data from a portable measurement unit according to a preferred embodiment of the preset invention;

FIG. 11A is a view showing a format of the measurement information data;

FIG. 11B is a view showing an example of downloaded measurement information data;

FIG. 12a and FIG. 12b are a flow chart showing a process in which a cradle is connected to a server and makes communication with the server with respect to the measurement information data according to a preferred embodiment of the present invention;

FIG. 13 is a flow chart representing a procedure of transferring data after a cradle is connected with a server according to a preferred embodiment of the present invention; and

FIG. 14 is a flow chart representing a procedure of transmitting data between a cradle and a server in more detail according to a preferred embodiment of the present invention.

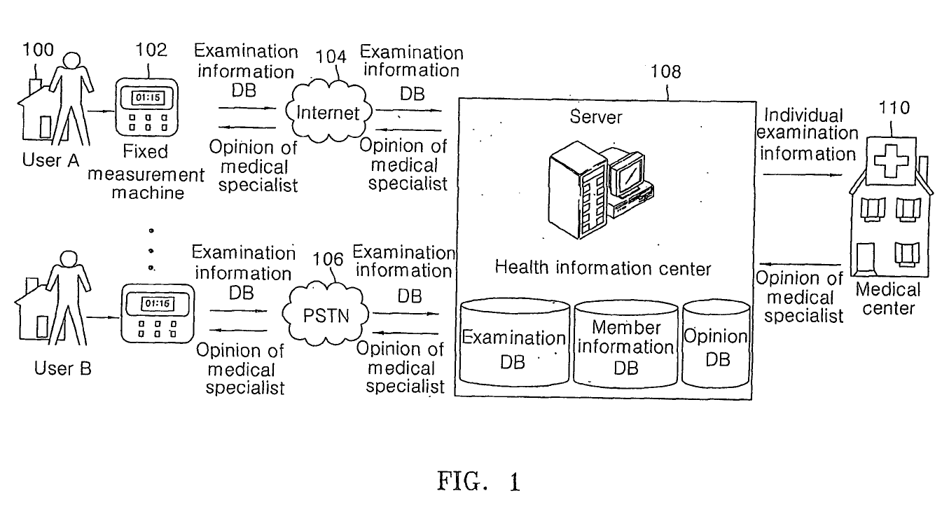

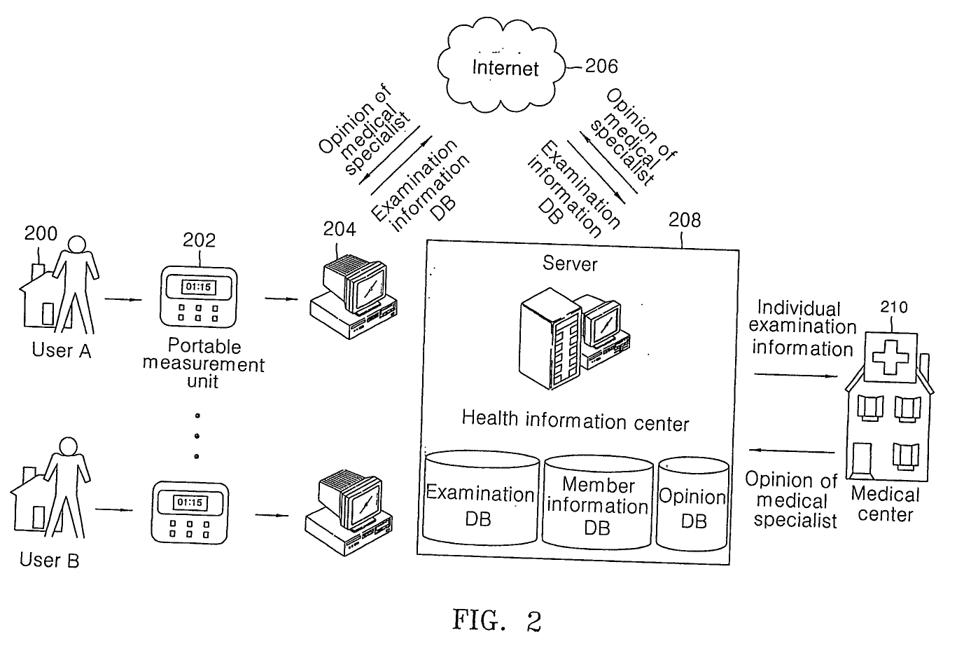

### BEST MODE FOR CARRYING OUT THE INVENTION

Reference will now be made in detail to the preferred embodiments of the present invention.

In the following description of the present invention, a detailed description of known functions and configurations incorporated herein will be omitted when it may make the subject matter of the present invention rather unclear.

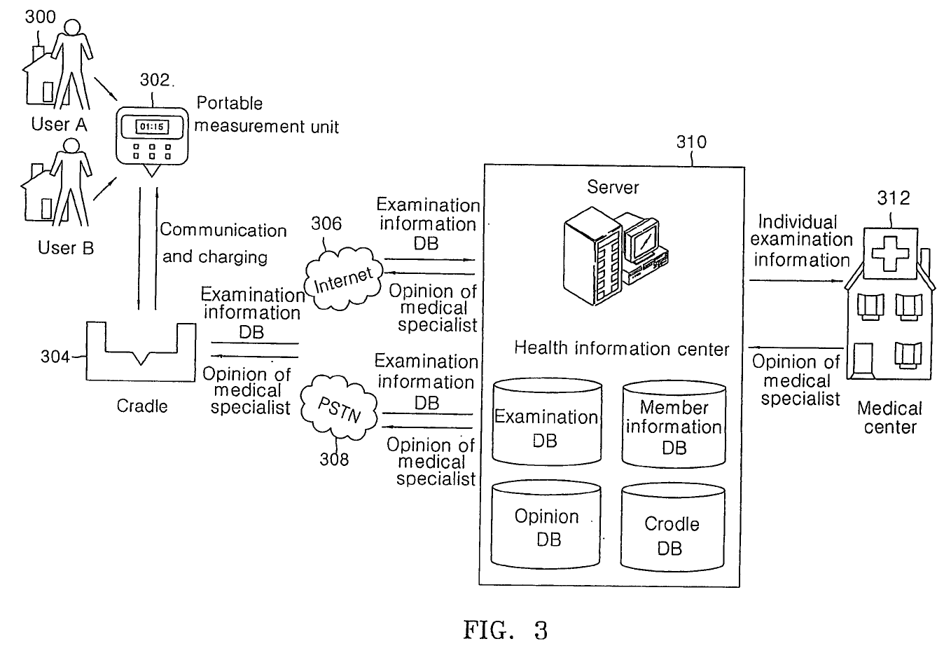

FIG. 3 is a schematic view showing a structure of a healthcare system according to a preferred embodiment of the present invention.

As shown in FIG. 3, the present invention includes a portable measurement unit 302, a cradle 304, and a server 310. Herein, the portable measurement unit 302 is used for a measurement operation for use in acquiring biological measurement data. The cradle 304 is used for performing operations such as storage, confirmation, and communication by receiving the biological measurement data from the portable measurement unit 302 through wired/wireless communication. The server 310 is connected with an information network such as the Internet 306, a LAN (not shown), and a PSTN 308 using TCP/IP, a modem, etc., so that the server 310 connects the cradle 304 to a medical specialist of a medical center such as a hospital, etc.

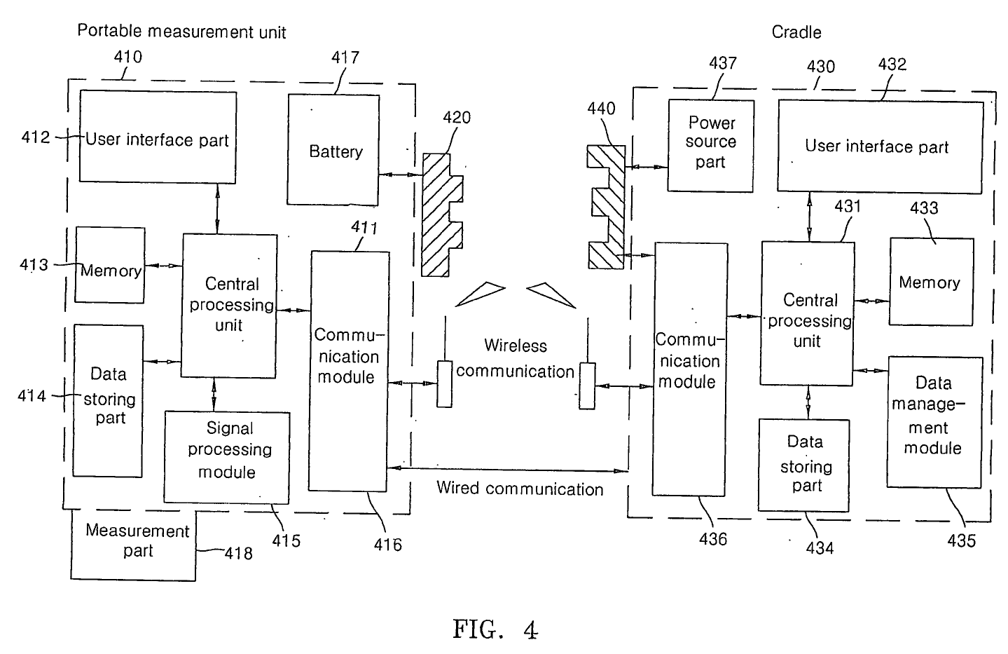

FIG. 4 is a view showing internal structures of the portable measurement unit 302 and the cradle 304 and a method of connecting the portable measurement unit 302 to the cradle 304 according to a preferred embodiment of the present invention.

A portable measurement unit 410 measures a required biological measurement data, temporarily stores the measured biological measurement data, classifies the stored biological measurement data according to characteristics of the stored biological measurement data, and performs data transmission/receipt through wired/wireless communication. Herein, characteristic classification of the stored biological measurement data signifies that the stored biological measurement data are classified according to characteristics of the stored biological measurement data in an order of priority. For example, when an urgent situation occurs with an uncomfortable patient so that a quick treatment is required for the patient, the portable measurement unit 410 can transfer the biological measurement data for the patient to a cradle 430 through wireless communication.

The portable measurement unit 410 includes a central processing unit 411, a user interface part 412, a memory 413, a data storing part 414, a signal processing module 415, a communication module 416, a battery 417, a measurement part 418, and a first communication port 420.

The central processing unit 411 controls functions of the portable measurement unit 410 and operates a measurement program for a measurement operation of the portable measurement unit 410.

The user interface part 412 includes a keypad consisting of a plurality of key buttons used for receiving a command or receiving the measured biological measurement data, and a display unit presenting the received data, letters in relation to measurement start or stop, measurement values, measurement progression, etc., and images, etc.

Also, if medical information such as opinion of a medical specialist received from the server 310, etc. is stored in the portable measurement unit 410, the user interface part. 412 displays the medical information. The medical information can be automatically presented before measuring of biological data or after measuring biological data. If there is medical information to be displayed, a display light is turned on or turned off to notify medical information to be displayed. Also, it is possible to display medical information by allowing a user to push a button. In addition, such medical information can be visually presented on a screen as letters or images or can be generated as sound information.

The memory 413 stores temporary data generated while operating the portable measurement unit 410 and is used as a storage unit for loading of a measurement program.

The data storing part 414 is used for storing a variety of biological measurement data measured by the portable measurement unit 410. The data stored in the data storing part 414 can be transferred to the cradle 430 through various methods such as wired/wireless communication through wired/wireless communication network, a cable communication using a coaxial cable, a direct communication using coupling of communication terminals.

The signal processing module 415 converts basic physical factors such as current, voltage, resistance, and so forth, which include biological measurement information such as blood pressure, blood sugar, and so forth, into required type data. Also, the signal processing module 415 has a specific measurement program based on a sort of an object to be measured by the portable measurement unit 410. That is, if the portable measurement unit 410 is a blood sugar measurement unit, the signal processing module 415 has a blood sugar measurement program. If the portable measurement unit 410 is a blood pressure measurement unit, the signal processing module 415 has a blood pressure measurement program. A measurement program is executed by an operation signal of the central processing unit 411 and is under the control of a user through the user interface part 412.

The communication module 416 transfers measurement information data, which include biological measurement data converted by the signal processing module 415, measurement time, an ID of the portable measurement unit 410, and a user ID, to the cradle 430 through wired/wireless communication. The communication module 416 has a communication device such as a radio frequency (RF) signal processing unit (not shown) used for transferring the measurement information data to wired/wireless communication network after receiving the measurement information data converted into digital data from analog data according to the control of the central processing unit 411. Also, the communication module 416 operates as a coupling unit connecting the portable measurement unit 410 to the cradle 430 together with the following first communication port 420.

A method of communication between the portable measurement unit 410 and the cradle 430 varies depending on types of data measured by the portable measurement unit 410. If the measured data are data to be treated urgently, the communication between the portable measurement unit 410 and the cradle 430 is achieved in wireless. Otherwise, the measured data can be transferred through wired communication. However, the present invention is not limited to these cases.

Herein, wireless communication methods used for an urgent situation include a wireless LAN method mainly used in a short distance, a Bluetooth method, a simple RF method, and an infrared communication method such as IrDA. The wireless communication methods are used for transferring the measurement information data to be urgently-treated to the cradle 430 from the portable measurement unit 410. Also, if necessary, the measurement information data to be urgently-treated can be directly transferred to the server 310 and not to the cradle 430 through a code division multiple access (CDMA) method or a global system for mobile communication (GSM) method used for a cellular phone. In addition, if the present invention employs wireless communication methods as described above, the present invention can be used for detecting an abnormal symptom of a patient who feels uncomfortable.

The measurement information data which need not be treated urgent may be transmitted through wired communication between the first communication port 420 of the portable measurement unit 410 and a second communication port 440 of the cradle 430, when the portable measurement unit 410 is connected to the cradle 430.

If a distance between the portable measurement unit 410 performing a measurement operation and the cradle 430 is short (under a few meters), the converted measurement information data can be transmitted/received by using a cable for communication. Herein, a serial communication cable, a parallel communication cable, a universal serial bus (USB) cable, and so on can be used as a cable for a short distance communication. In order to employ the cable for short distance communication, the communication module 416 may have ports for connecting the above communication cables to the communication module 416. That is, the communication module 416 may have a serial communication port, a parallel communication port, and a USB communication port.

The battery 417 may be a rechargeable battery supplying power for operation of the portable measurement unit 410. However, the battery 417 is not limited to the rechargeable battery.

The measurement part 418 is connected or exposed to a human body to perform a sensing function, etc.

The first communication port 420 of the portable measurement unit 410 is coupled with the second communication port 440 formed in the cradle 430 so as to deliver the measurement information data or receive power. That is, power delivered through the first communication port 420 is used for charging the battery 417. Herein, it is possible to use the battery 417 alone as a power source without receiving power through the first communication port 420.

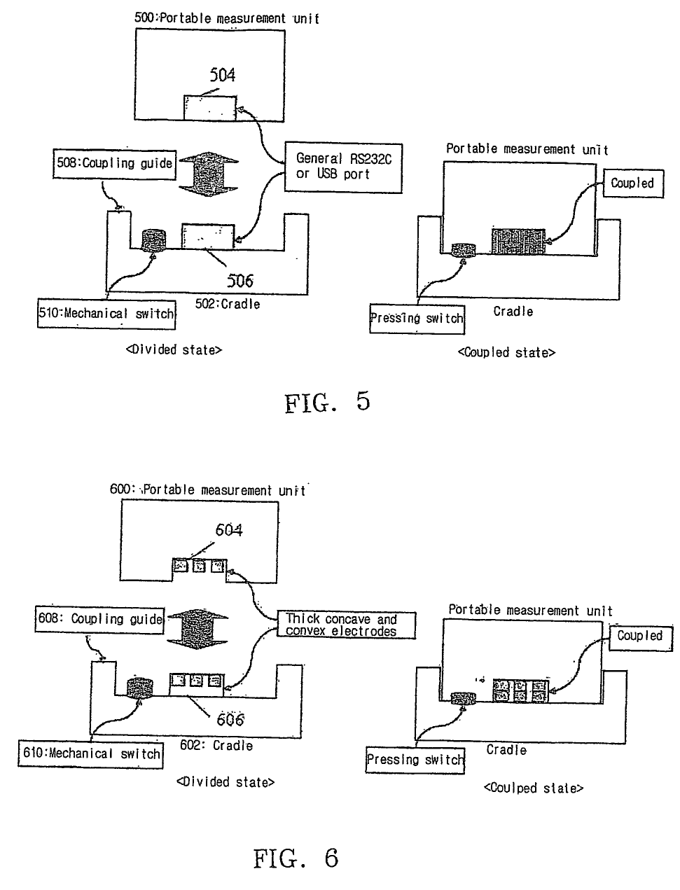

There are four structures allowing the portable measurement unit 410 and the cradle 430 to make communication with each other by using the first communication port 420 and the second communication port 440 as shown in FIGS. 5 to 8.

FIG. 5 is a view showing a structure for coupling the portable measurement unit 410 with the cradle 430 through a communication port according to a preferred embodiment of the present invention.

A first communication port 504 of a portable measurement unit 500 and a second communication port 506 of a cradle 502 must be structured in such a manner that the first communication port 504 is easily coupled with the second communication port 506. If the cradle 502 and the portable measurement unit 500 employ a communication method of RS232C or USB, the first communication port 504 and the second communication port 506 have a general shape of an RS232C port or an USB port. Also, as shown in FIG. 5, coupling guides 508 are stood around the cradle 502 and a mechanical switch 510 is installed on the cradle 502 in such a manner that if the portable measurement unit 500 is coupled with the cradle 502, the mechanical switch is pushed in order to confirm coupling of two pieces of equipment.

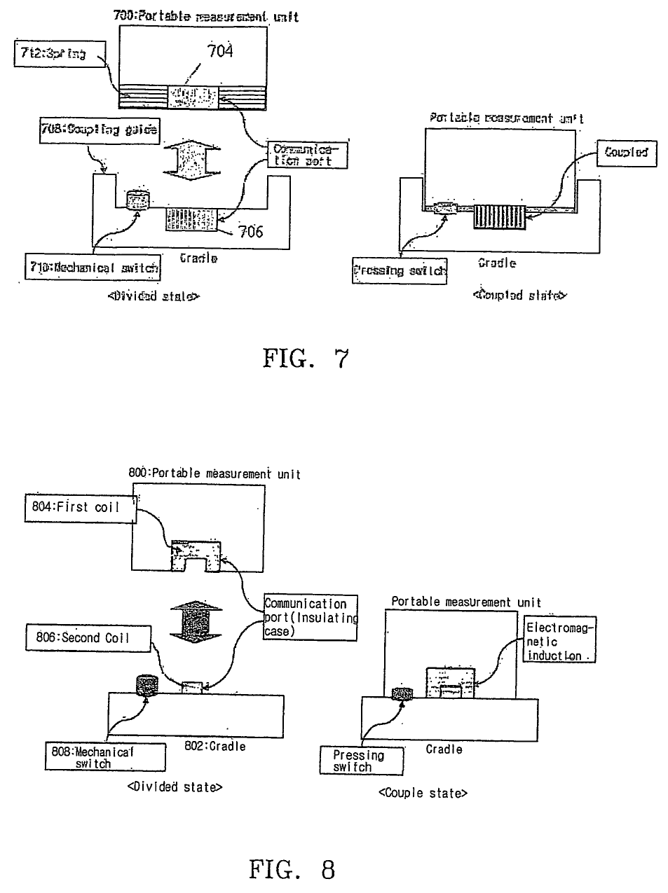

FIG. 6 is a view showing a structure of coupling the portable measurement unit 410 with the cradle 430 by means of concave and convex electrodes.

In case of the RS232C port or the USB port described above, pins can be modified or stability of coupling may be reduced. Therefore, as shown in FIG. 6, a first communication port 604 of a portable measurement unit 600 can be coupled with a second communication port 606 of a cradle 602 by using thicker concave and convex electrodes. Similar to FIG. 5, according to the structure shown in FIG. 6, coupling guides are installed around the cradle 602 and a mechanical switch 610 can be used. If the concave and convex electrodes operate as the coupling guides 608, it is unnecessary to stand the coupling guides 608.

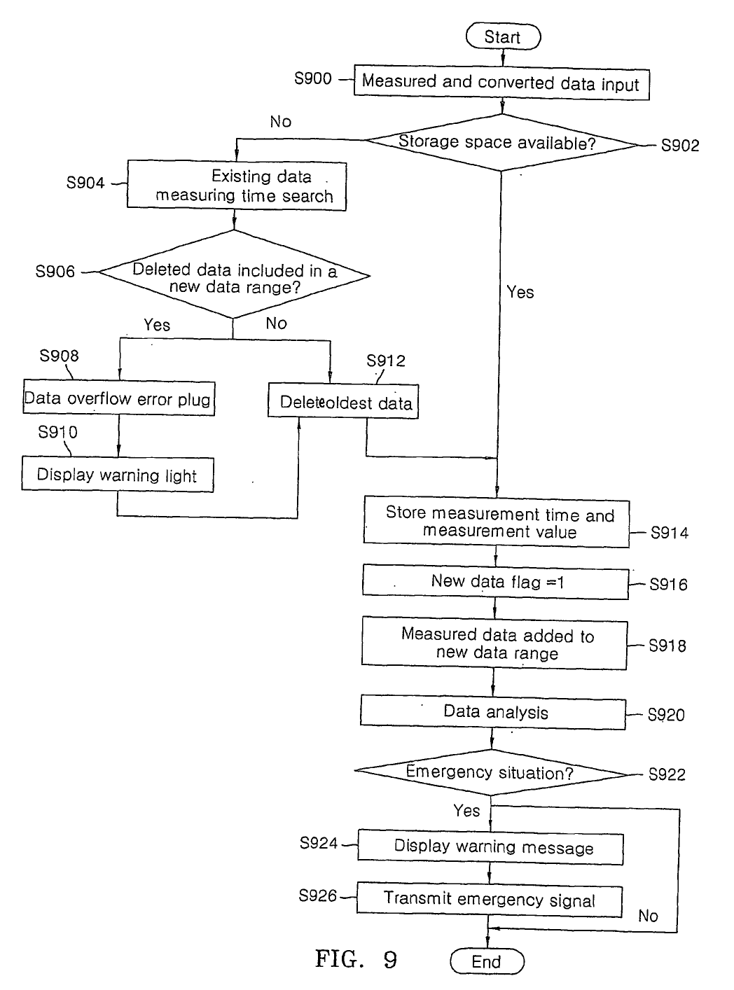

FIG. 7 is a view showing a structure in which the first communication port 420 and the second communication port 440 are exposed only when they are coupled with each other.

As shown in FIG. 7, when the portable measurement unit 700 conducts communication with the cradle 702, a first communication port 704 of the portable measurement unit 700 and a second communication port 706 of the cradle are not exposed to the outside thereof ordinarily but exposed to the outside thereof so as to be coupled with each other only when they are coupled with each other. This method employs a coupling guide 708 and a mechanical switch 710 like the above-described methods. However, unlike the above-described methods, the portable measurement unit 700 is equipped with a spring 712, so that the first communication port 704 and the second communication port 706 are exposed only when they are coupled with each other.

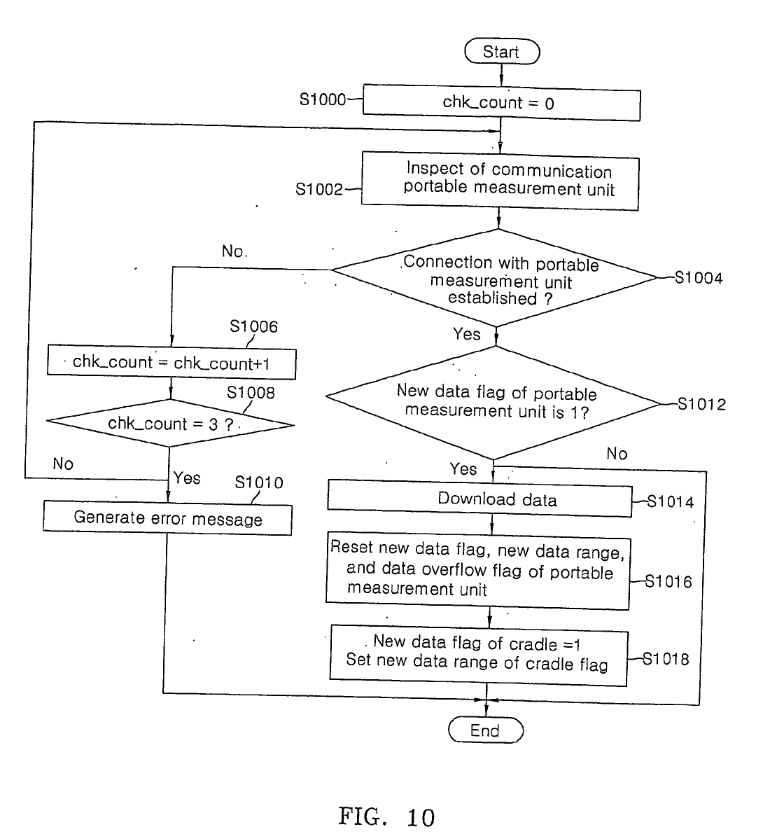

FIG. 8 is a view showing a structure in which the first communication port 420 and the second communication port 440 are coupled with each other without direct and electric contact according to a preferred embodiment of the present invention.

As shown in FIG. 8, when the portable measurement unit 800 is coupled with the cradle 802, a first communication port of the portable measurement unit and a second communication port of the cradle transfer data to each other through a first coil 804 and a second coil 806 by means of electromagnetic induction rather than electric conduction. In FIG. 8, although a mechanical switch 808 is used, an additional coupling guide is not required since the first and the second communication ports perform a function of the coupling guide.

Meanwhile, there are four charging methods achieved when the portable measurement unit 410 and the cradle 430 are coupled with each other.

The portable measurement unit 410 according to a preferred embodiment of the present invention includes a blood sugar measurement unit, a pulse measurement unit, a blood pressure measurement unit, a body fat analysis unit, an electrocardiogram measurement unit, a brain wave measurement unit, a respiration measurement unit, an SpO₂ measurement unit, a blood analysis unit, and a urine analysis unit. Also, biological measurement according to a preferred embodiment of the present invention includes blood sugar measurement, pulse measurement, blood pressure measurement, body fat measurement, respiration measurement, SpO₂ measurement, blood analysis, and urine analysis.

In an initial state of the portable measurement unit 410, there are no biological measurement data. In this state, a new data flag and a data overflow error flag of the portable measurement unit 410 have a value of “0”. Also, a new data range of the portable measurement unit 410 is not established. Herein, the new data flag of the portable measurement unit 410 represents whether or not new data to be transferred to the cradle 430 from among biological measurement data measured and converted by the portable measurement unit 410, exist. The data overflow error flag is set when the amount of data is larger than the size of a storage space of the portable measurement unit. The setting of the data overflow error flag refers to the fact that newly measured data are deleted and not normally transferred to the cradle. Also, the new data range of the portable measurement unit 410 is a value representing a position of new data to be transferred to the cradle 430, and can specify a physical or a logical memory address.

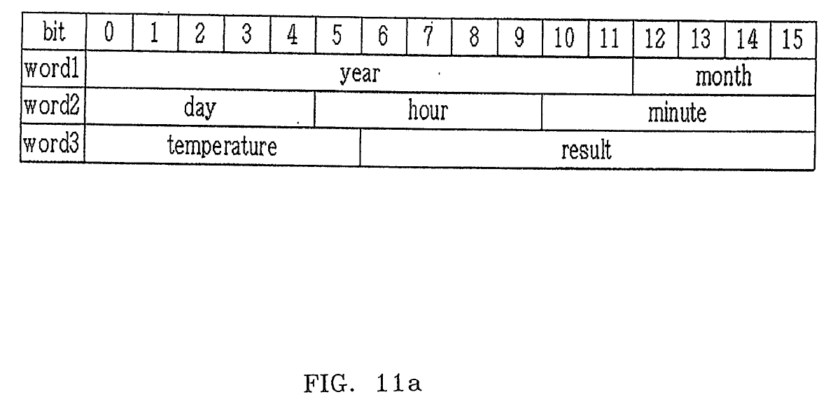

FIG. 9 is a flow chart showing an operation of the portable measurement unit 410 according to a preferred embodiment of the present invention.

When the measurement part 418 of the portable measurement unit 410 measures biological measurement data and the signal processing module 415 obtains converted biological measurement data (S900), the central processing unit 411 of the portable measurement unit 410 determines whether or not a storage has an available space in the data storing part 414 (S902). If a storage has an available space, measurement time of new data and biological measurement data are stored in the data storing part 414 of the portable measurement unit 410 (S914). If there is no storage space available, the portable measurement unit 410 checks measurement time of already stored data (S904). Thereafter, the portable measurement unit 410 determines whether or not data to be deleted (i.e., data stored for the longest time) exist within the new data range of the portable measurement unit 410 (S906). If the data to be deleted exist within the new data range of the portable measurement unit 410, a new data value is not normally downloaded to the cradle 430. Therefore, the data overflow error flag is set as a value of “1” (S908), and a warning light is turned on (S910). Thereafter, if the data to be deleted does not exist within the new data range of the portable measurement unit 410, the data stored for the longest time are deleted and then measurement time of new data and biological measurement data are stored in the position of the deleted data (S914). The new data flag of the portable measurement unit 410 is set as “1” after storing the measurement time of the new data and the biological measurement data (S916). Also, newly measured data range is added to the new data range of the portable measurement unit 410 (S918).

The portable measurement unit 410 has an additional function of coping with an emergency by analyzing the biological measurement data. That is, steps following step 920 are selectively carried out in order to deal with an emergency. The portable measurement unit 410 analyzes the measured data (S920) so as to determine whether or not an emergency occurs (S922). If it is determined that an emergency occurs, the portable measurement unit 410 displays warning messages together with a warning light and/or a warning sound (S924). Also, a communication module 416 of the portable measurement unit 410 transmits an emergency indicating signal to the cradle 430 through wireless communication. The cradle 430, which has received the emergency indicating signal, transmits the emergency indicating signal to the server 310 or an emergency server (not shown) (S926). Meanwhile, if necessary, the portable measurement unit 410 may directly transmit an emergency signal to the server 310 or the emergency server (not shown) without passing through the cradle 430. In this state, mobile communication modules such as CDMA, GSM, etc. must be accommodated in the portable measurement unit 410, and address of the server 310 or the emergency server (not shown) to be connected to the portable measurement unit 410 in case of an emergency must be stored in the portable measurement unit 410. Herein, the emergency server (not shown) is a server having an address designated in order to perform high-reliability communication. Also, the emergency server (not shown) is a sever additionally installed in order to prevent communication failure resulting from such as “busy” state, etc. when an emergency occurs. Also, if the emergency server (not shown) does not exist, the general server 310 additionally has an address desigated in order to perform high-reliability communication in case of an emergency so as to cope with an emergency. Step 926 will further be described as of describing data transmission/receipt in case of an emergency.

An internal structure of the cradle 430 according to a preferred embodiment of the present invention basically may include a central processing unit 431, a memory 433, a data storing part 434, a communication module 436, a power source part 437, and a communication port 440 of the cradle, and additionally include a user interface part 432 and a data management module 435. Hereinafter, for the purpose of illustration, internal parts of the cradle 430 having functions similar to functions of the portable measurement unit 410 will not be described in detail.

The user interface part 432 includes a key pad having a plurality of key buttons and a display unit, wherein the key buttons allows users to receive measurement information data from the portable measurement unit 410 or to perform operations such as analysis, manufacturing, etc., with respect to received and stored measurement information data. If the cradle 430 according to a preferred embodiment of the present is designed to automatically transfer data received from the portable measurement unit 430, the key buttons may be omitted from the user interface part 432.

The data management module 435 includes a predetermined data management program for performing operations such as analysis, manufacturing, etc. with respect to measurement information data stored in the data storing part 434.

The cradle 430 according to a preferred embodiment of the present invention additionally has a function of charging the battery 417 by allowing current supplied from the power source part 437 of the cradle 430 to deliver to the battery 417 of the portable measurement unit 410, if the first communication port 420 of the portable measurement unit 410 and the second communication port 440 of the cradle 430 are connected to each other through methods described with reference to FIGS. 5 to 8.

Meanwhile, the cradle 430 according to a preferred embodiment of the present invention can be used for one or more portable measurement units 410 having different services. That is, the cradle 430 receives and stores measurement information data including blood sugar measuring data, pulse measuring data, blood pressure measuring data, body fat analyzing data, electrocardiogram measuring data, brain wave measuring data, respiration measuring data, SpO₂ measuring data, blood analyzing data, and urine analyzing data, and transmits and receives stored measurement information data.

The communication module 436 of the cradle 430 according to a preferred embodiment of the present invention is similar to the communication module 416 of the portable measurement unit 410. However, the communication module 436 of the cradle 430 requires a module capable of making communication with the server 310. According to a preferred embodiment of the present invention, the cradle 430 includes a modem so as to be connected to the PSTN 308 or includes a LAN card and adapts TCP/IP so as to be connected to the Internet 306, so that the cradle 430 makes communication with the server 310. The communication module 436 operates as a coupling unit connecting the cradle 430 to the portable measurement unit 410 or the cradle 430 to the server 310.

According to a preferred embodiment of the present invention, the cradle 430 makes data communication with the server 310 on the basis of dual tone multi-frequency (DTMF). Herein, the DTMF is used for synthesizing two different frequencies and generating signals so as to perform communication. Also, the DTMF is basically used for an electronic telephone or a tone telephone. The DTMF is mainly used for an electromagnetic switch. Also, when pushing corresponding numbers of a telephone, signals having dual multi-frequency are applied to a switching system and the switching system interprets the signals so as to convert the interpreted signals into digit information.

Table 1 represents an example of matching synthesized signals with frequencies used for a preferred embodiment of the present invention.

TABLE 1

High

| Low | 1209 Hz | 1336 Hz | 1477 Hz | 1633 Hz |
|---|---|---|---|---|
| 697 Hz | 1 | 2 | 3 | A |
| 770 Hz | 4 | 5 | 6 | B |
| 852 Hz | 7 | 8 | 9 | C |
| 941 Hz | * | 0 | # | D |

As represented in Table 1, according to a preferred embodiment of the present invention, the cradle 430 makes communication with the server 310 using numerals 0 to 9 generated based on the DTMF signals and using “A”, “B”, “C”, “D” “*”, and “#” as communication control codes. Herein, since “A”, “B”, “C”, “D” “*”, and “#” are not included on a dial pad of a ordinary telephone, signals generated through “A”, “B”, “C”, “D” “*”, and “#” are not generated by the ordinary telephone. Therefore, according to a preferred embodiment of the present invention, since the signals generated through “A”, “B”, “C”, “D” “*”, and “#” are used as communication control signals, it is possible to reduce communication errors due to cross talk, etc. Meanwhile, according to a preferred embodiment of the present invention, data transmission of the DTMF signals is achieved through a decimal numeral.

Meanwhile, data processed by the cradle 430 according to a preferred embodiment of the present invention include measurement information data, environment data, flag data, etc.

The measurement information data according to a preferred embodiment of the present invention include a user ID, an ID of the portable measurement unit 410, measurement time, and biological measurement data converted by the signal processing module 415 of the portable measurement unit 410. When the cradle 430 makes communication with the server 310, if the cradle 430 sends a user ID and an ID of the portable measurement unit 410 to the server 310, the server 310 delivers diagnosis information corresponding to a user stored therein to the cradle 430 using the user ID and the ID of the portable measurement unit 410. Accordingly, it is possible to provide suitable services. Herein, the reason requiring the ID of the portable measurement unit 410 as well as an ID of the cradle 430 is that one cradle 430 may be connected to a plurality of portable measurement units 410. In this case, since the cradle 430 can make sufficient communication with the server by using only the ID of the portable measurement unit 410, it is unnecessary for the cradle 430 to have an ID. Also, when one portable measurement unit 410 is used by several users, user IDs are stored in the portable measurement unit 410 and the server 310 and the users can use services, if each user has a corresponding portable measurement unit 410.

The environment data according to a preferred embodiment of the present invention includes an address (IP address or telephone number) of a server 310 which will receive the measurement information data and transmission time to be used for transmitting the measurement information data. Also, the environment data includes an address of the emergency server (not shown) for providing communication having high reliability in case of an emergency. The environment data are specified as initial values when the cradle 430 is produced but can be modified by making communication with the server 310.

The environment data of the cradle 430 can be remotely set. The remote setting of the environment data means that a telephone number or an IP address of the server 310 to be connected to the cradle 430 and time used for making communication with the server 310 are set remotely. Also, when the cradle 430 is initially connected to the server 310 or circumference conditions are changed, related environment data can be remotely modified.

The flag data according to a preferred embodiment of the present invention include new data flag of the cradle 430, new data range of the cradle 430, and the other error flags. The new data flag of the cradle 430 represents whether or not new data to be transmitted to the server 310 exist. The new data range of the cradle 430 is a data value representing a position of new data to be transmitted to the server 310 and can represent a physical or a logical address. Also, the other error flags are set when measurement information data and diagnosis information are not normally transmitted/received between the cradle 430 and the portable measurement unit 410 or between the cradle 430 and the server 310.

In an initial state of the cradle 430, there are no data, the new data flag of the cradle 430 and the error flags are set as “0”, and the new data range of the cradle 430 does not exist.

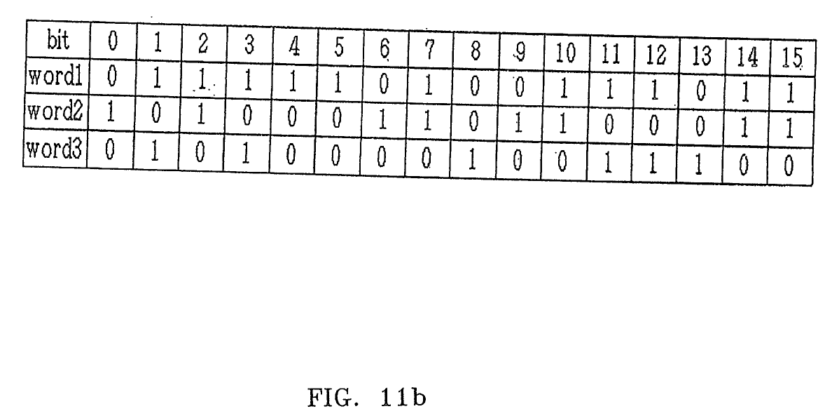

FIG. 10 is a flow chart representing a process in which the cradle 430 downloads data, which represent cases requiring no-emergency treatment, from the portable measurement unit 410 according to a preferred embodiment of the present invention.

FIG. 10 shows a data download process in case of ordinary times and not in case of an emergency. A description about a data download process of an emergency situation will be omitted here but given below.

First, when the cradle 430 is coupled with the portable measurement unit 410, the cradle 430 must check whether or not communication initial establishment for starting communication is achieved. As a check method, there is a method of checking the communication initial establishment by operating mechanical switches 510, 610, 710, and 808 performing pushing operation as shown in FIGS. 5 to 8 if the portable measurement unit 410 is coupled with the cradle 430, a method of checking the communication initial establishment by performing an electrical check by a predetermined time interval, or a method of checking the communication initial establishment by performing the electrical check after operating the mechanical switches with a mixture of the two above methods. If the communication initial establishment for starting communication is checked, the cradle 430 tries to download measurement information data. Herein, when the portable measurement unit 410 is coupled with the cradle 430, the measurement information data can be automatically downloaded through a program included in the cradle 410.

In a procedure of checking the communication initial establishment, a variable called “chk_count” is used for a process of handling errors when communication failure occurs. If the communication initial establishment is checked, a value of “chk_count” is initialized as “0” (S1000). Thereafter, the communication module 436 of the cradle 430 inspects a communication initial establishment between the portable measurement unit 410 and the cradle 430 (S1002).

Then, it is determined that communication between the portable measurement unit 410 and the cradle 430 is established (S1004). If communication fails, it is checked that communication is established after a predetermined time lapses. In this process, if the communication fails three times in a row, the central processing unit 431 of the cradle 430 generates a communication failure error message and terminates the process. In FIG. 10, a check procedure corresponds to step 1006 and step 1008. In step 1008, “3” marked on a box can be changed. If communication is successfully established, a value of the new data flag of the portable measurement unit 410 is checked (S1012). If the value of the new data flag of the portable measurement unit 410 is “1”, new data exist, so that measurement information data are downloaded to the cradle 430 (S1014).

If the measurement information data are downloaded, the new data flag, the new data range, and the data overflow error flag of the portable measurement unit 410 are reset (S1016). Also, the data management module 435 of the cradle 430 sets the new data flag of the cradle 430 as “1” and the new data range of the cradle 430 is established (S1018). A procedure (not shown) of inspecting whether or not a storage space required when downloading and storing data remains is basically the same as the inspection procedure of the portable measurement unit 410 described with reference to FIG. 9.

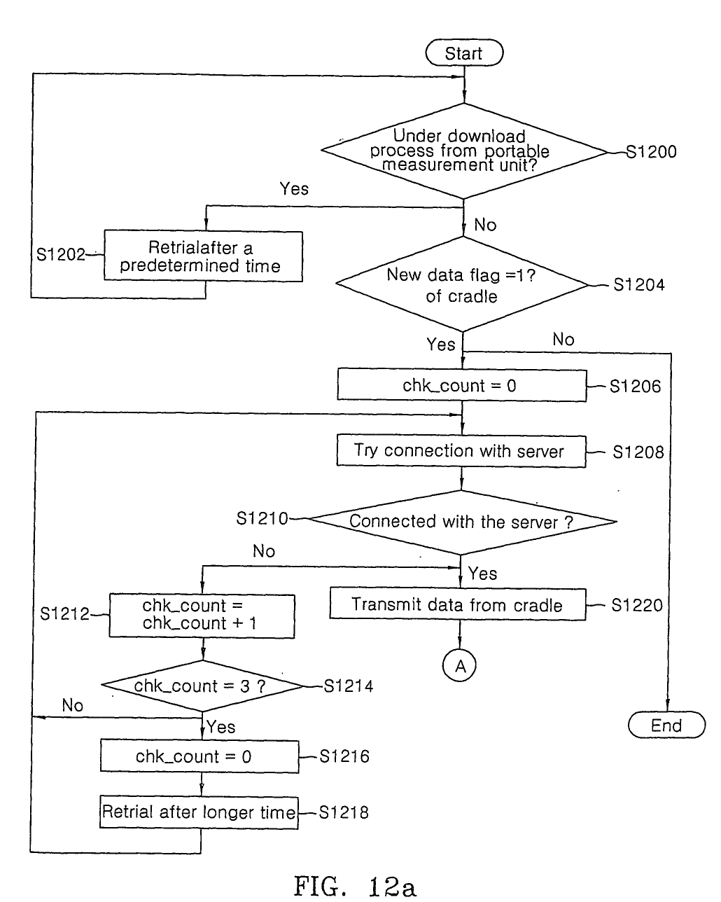

FIG. 11A is a view showing a format of the measurement information data, and FIG. 11B is a view showing one embodiment of downloaded measurement information data.

As shown in FIG. 11A, the downloaded measurement information data consist of three words. A first word has “year” and “month”, a second word has “measurement data” and “measurement time”, and a third word has “measurement temperature” and “measurement result”. FIG. 11B shows an example of the measurement information data in which the measurement date is Nov. 20, 2003, the measurement time is 1:35 P.M, a measurement temperature is 20° C., and a measurement result is information of 156. Since data transmitted/received between the portable measurement unit 410 and the cradle 430 is digital, “The year 2003” is represented as “011111010011” on fields between “0” bit and 11 “bit” of the first word, “November” is represented as “1011” on fields between “12” bit and “15” bit of the first word, “20^{th} day” is represented as “10100” on fields between “0” bit and “4” bit of the second word. “13 hours” is represented as “01101” on fields between “5” bit and “9” bit of the second word, “35 minutes” is represented as “100011” on fields between “10” bit and “15” bit of the first word, “20° C”. is represented as “010100” on fields between “0” bit, “5” bit of the third word, and “156” is represented as “0010011100” on fields between “6” bit and “15” bit of the third word.

A method of transmitting data from the cradle 430 to the portable measurement unit 410 is basically identical to the method of transmitting the measurement information data from the portable measurement unit 410 to the cradle 430. Also, the characteristics of related parameters are basically the same with each other. For example, if a new medical information flag of the portable measurement unit 410 is established and new medical information to be presented exits, the new medical information flag is set as “1”. If the new medical information flag is “1”, the portable measurement unit 410 turns off an indication light. The above operations are sequentially performed. If the cradle 430 transmits new medical information to the portable measurement unit 410, the portable measurement unit 410 stores the new medical information and modifies related variables.

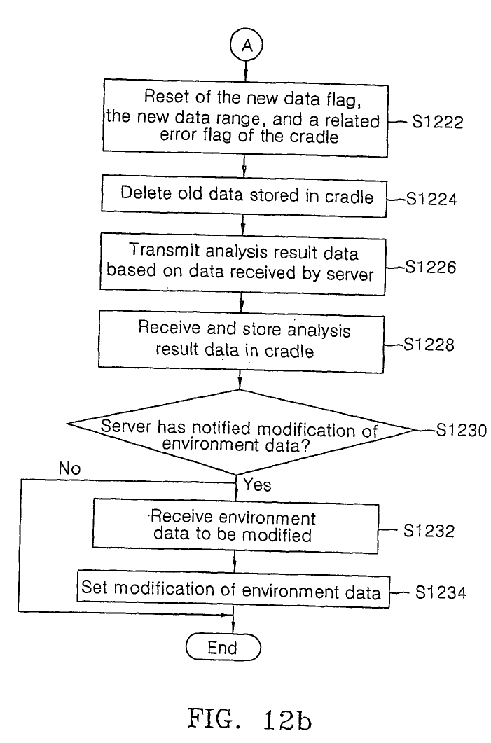

FIG. 12a and FIG. 12b are a flow chart showing a process in which the cradle 430 is connected to the server 310 and makes communication with the server 310 with respect to the measurement information data.

First, if data transmission time set in the cradle 430 lapses, it is determined whether or not the cradle 430 downloads data from the portable measurement unit 410 (S1200). If the cradle 430 is downloading data from the portable measurement unit 410, connection is retried after a predetermined time (S1202). If the cradle 430 does not download data from the portable measurement unit 410, a value of the new data flag of the cradle 430 is determined (S1204). If the value of the new data flag is “1”, new data exist, so that the communication module 436 of the cradle 430 tries a connection with a predetermined server 310 (S1208). Herein, in order to perform a retrial process if the connection fails at step 1208, a variable of “chk₋count” is used (S1206).

Meanwhile, according to a preferred embodiment of the present invention, in addition to a predetermined time, the cradle 430 can be automatically connected to the server 310 through a program included in the cradle 430 immediately after the portable measurement unit 410 is coupled with the cradle 430.

After the communication module 436 of the cradle 430 determines whether or not the cradle 430 is connected with the server 310, if connection fails due to a “busy” state, disconnection after call connection, etc., the cradle 430 retries connection with the server 310 after a predetermined time. If connection fails three times in series, the cradle 430 retries such connection with the server 310 after waiting for a predetermined period of time which is longer than previous time (S1218).

Meanwhile, if the cradle 430 is successfully connected with the server 310, the cradle 430 transfers measurement information data such as biological measurement data, measurement time, an ID of the portable measurement unit 410 to the server 310 (S1220), and the new data flag, the new data range, and a related error flag of the cradle 430 are reset (S1222). After the central processing unit 431 of the cradle 430 searches for storage time of data stored in the cradle 430 with considering the total storage space, the central processing unit 431 deletes data stored for the longest time from among data stored in the cradle 430 (S1224). Herein, some data can remain in order to prevent data loss resulting from faults of the server 310.

The server 310 transmits analysis result data based on the currently or previously received measurement information data to the cradle 430 (S1226), and the communication module 436 of the cradle 430 receives the analysis result data and stores the analysis result data in the data storing part 434 (S1228). Finally, the communication module 436 of the cradle 430 receives the analysis result data and then determines whether or not modification of the environment data such as a server address or transmission time is reported (S1232). If the modification of the environment data is reported, the cradle 430 receives the modified environment data (S1232) and modifies the environment data establishment (S1234). Herein, an order of receiving the environment data and the analysis result data can be exchanged. Also, operations (e.g., deletion of data stored in the cradle 430), which are independently performed by the cradle 430, can be carried out after connection release. Also, nevertheless the cradle 430 has no data to be transmitted to the server 310, the cradle 430 can be connected with the server 310 in order to receive the analysis data.

As described above, if there is a function of directly connecting the portable measurement unit 410 to the server 310 without passing through the cradle 430 in case of an emergency, related environment data must be transferred to the portable measurement unit 410. For example, when an address of the server 310 to be connected with the portable measurement unit 410 is changed, the cradle 430 transfers the changed server address to the portable measurement unit 410 and the portable measurement unit 430 replaces a server address thereof with the changed server address.

The server 310, which has downloaded data, performs inspection and modification with respect to overlap of data and checks and analyzes an entire communication state, thereby optimizing connection time of each client in order to increase system efficiency. In this state, the server 310, which memorizes related information, transfers information such as changed address of the server 310, transmission time of the measurement information data, etc., to the cradle 430 when the cradle 430 is connected with the server 310, thereby re-establishing the environment data of the cradle 430.

If necessary, after the measurement information data are distributed and received to several servers on the basis of an ID of the portable measurement unit 410 or an ID of a user, a central server can integrally manage the several servers. That is, after the cradle 430 transmits data to distribution servers through telephone lines, the distribution servers transmit data to the central server through the Internet.

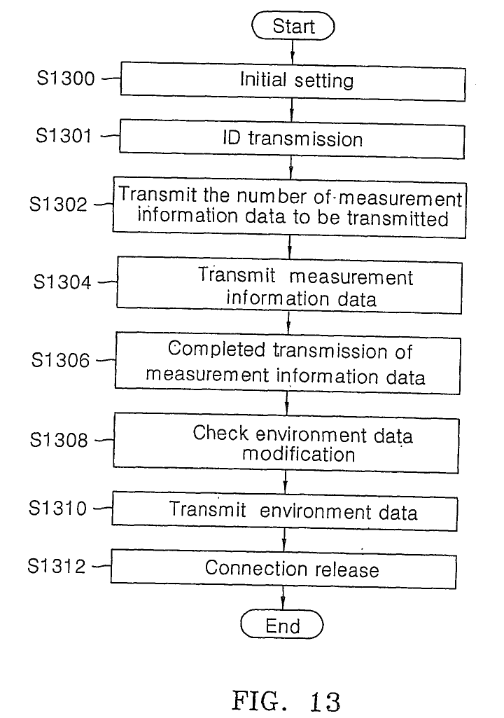

FIG. 13 is a flow chart representing a procedure (after step 1220) of transferring data after the cradle 430 is connected with the server 310.

Fist, if the cradle 430 notifies the server 310 of transmission start, the server 310 confirms data receipt start. The cradle 430 has completed an initial setting by re-confirming the transmission start (S1300). After the initial setting, the cradle 430 transfers an ID of the portable measurement unit 410 and an ID of a user to the server 310 (S1301).

Thereafter, the cradle 430 transfers the number of data to the server 310, and the server 310 checks the number of the data (s1302). Subsequently, the cradle 430 transfers a bundle of measurement data, which is a bundle of sequence numbers and content of the measurement information data, to the server 310, and the server 310 confirms the sequence number of the measurement information data (S1304). Herein, depending on the transmission speed, several bundles of measurement data may be grouped so as to be transferred to the server 310 as a package, or a bundle of measurement data may be divided into several parts so as to be transferred to the server 310. Herein, the measurement information data includes an ID of the portable measurement unit 410, an ID of a user, measurement time, biological measurement data, etc. However, if the ID of the portable measurement unit 410 and the ID of the user have been transferred at step 1301, corresponding ID information may be omitted from the measurement information data.

When the cradle 430 has completed data transmission, the cradle 430 notifies the server 310 of completion of the data transmission, and the server 310 confirms completion of data receipt (S1306). Thereafter, the cradle 430 inquires about whether or not the cradle 430 modifies environment data. The server 310 notifies the cradle 430 of whether or not environment data to be modified exist, and the cradle 430 checks whether or not the cradle 430 modifies the environment data on the basis of information notified from the server 310 (S1308). The server 310 transmits the number and types of the environment data to be modified to the cradle 430 if the environment data exist. At this time, the cradle 430 confirms the number of the environment data to be modified and the sorts of the environment data to be modified, and the server 310 transmits corresponding environment data to the cradle 430.

Thereafter, the cradle 430 confirms receipt of the environment data to be modified. Herein, if the environment data to be modified cannot be transferred as one packet, the environment data to be modified are divided into several groups for transmission. Herein, the server 310 assigns sequence numbers to the divided packets and transmits the total number of the divided packets to the cradle 430. Thereafter, a confirmation procedure is achieved (S1310). Lastly, the cradle 430 notifies the server 310 of connection release, and the server 310 confirms the connection release (S1312). Each process described above has specified time required for awaiting responses and specified number of retrials. If an error message to be transferred from the server 310 exists, corresponding processes may be added. Meanwhile, if the server 310 transmits medical information such as diagnosis information, etc., to the cradle 430, a process of transmitting the medical information is basically identical to the process of transmitting the above described environment data.

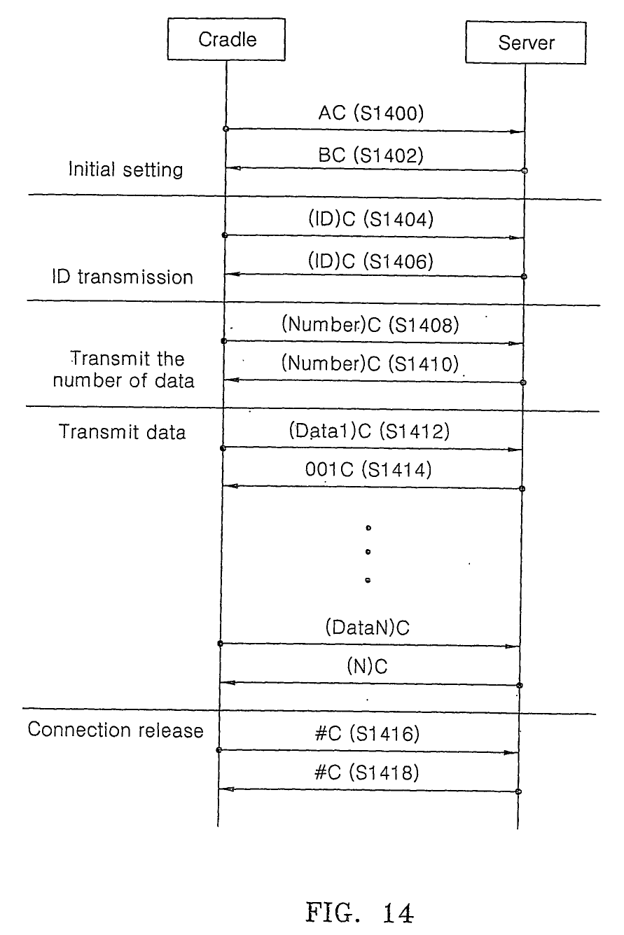

FIG. 14 is a flow chart representing a procedure of transmitting data between the cradle 430 and the server 310 in more detail according to a preferred embodiment of the present invention.

Hereinafter, the initial setting of step 1300 shown in FIG. 13 will be described in more detail. If the cradle 430 telephones the server 310 and connection is achieved, the cradle 430 transfers “AC” code to the server 310 (S1400). If the server 310 transfers “BC” code to the cradle 430 in response to the “AC” code (S1402), the initial setting has been completed. If a problem occurs in communication of the cradle 430 and the server 310, the server 310 transfers “DC” code, which is an error code, to the cradle 430. When an error occurs, retrials are performed for as many times as the specified number of retrials. If an error occurs after performing retrials, the connection is forcefully terminated. An error handling method to be described below is the same as the error handling method described above.

After the initial setting, an ID of the portable measurement unit 430 or an ID of a user is transferred (S1404). Hereinafter, it is assumed that one cradle 430 corresponds to one portable measurement unit 410. For example, if an ID is “123456”, the cradle 430 transfers “1234561C” to the server 310. The last letter “C” in “1234561C” refers to the end of transmission data. Also, “1” next to “C” is a kind of parity bit for use in checking errors and is a remainder obtained after dividing the sum of all data to be transmitted by 10. After the server 310 receives the ID, the server 310 checks a length of the received data and a parity bit of the received data. If the Id has been successfully transferred, the server 310 replies to the cradle 430 by transferring the received ID to the cradle 430 (S1406). After the cradle 430 receives the received ID from the server 310, the cradle 430 determines whether or not the transferred ID is equal to the received ID, so that the cradle 430 determines whether or not the ID has been successfully transmitted. Error handling is the same as the above described error handling in the initial setting step.

After the ID of the cradle 430 has transmitted, the cradle 430 transmits the number of data to be transmitted to the server 310 (S1408). A method of transmitting the number of data to be transmitted is identical to the method of transmitting the ID. For example, if the number of data to be transmitted is 12, the cradle 430 transmits “123C” and receives a response signal from the server 310 (S1410). Error detection can be achieved by a manner using a length or a parity bit, which is identical to the manner performed when transmitting the ID.

Thereafter, the cradle 430 transfers measurement information data to the sever 310 (S1412). For example, if a data value measured at 12:30Jan. 1, 2003(a temperature is 20° C. when measuring data) is 156, “200301011230 20 156 5 C” is transmitted. Herein, the spaces in “200301011230 20 156 5 C” are not actually marked in “200301011230 20 156 5 C” but have been inserted in “200301011230 20 156 5 C” for the purpose of description. In addition, since real measurement information data and transmission quality are very important, it is possible to transfer the measurement information data by adding redundancy factor information to existing parity bits. Herein, an error correction code used is may be any one of block codes such as Hamming Code, Cyclic Redundancy Check (CRC), etc., Convolutional Code, Concatenated Code, etc. According to a preferred embodiment of the present invention, the server 310 can perform error detection and error correction by using the error correction codes. If the server 310 successfully receives data, the server 310 transfers information about the number of received data to the cradle 430 (S1414). For example, if the server 310 receives second data, the server 310 transfers “002C”. The cradle 430 receives a signal like “002C” and checks whether or not an error has occurred.

If data transmission has been completed, the cradle 430 and the server 310 respectively give and receive a “#C” signal, which is a connection release signal, (S1416 and S 1418), so that communication is completed. The connection release signal can be used when the connection is forcefully terminated due to an occurrence of an error.

The above description is directed to a method of transmitting/receiving the measurement information data. Transmission/receipt of the environment data may be performed in the same method as used for transmitting/receiving the measurement information data.

Meanwhile, the server 310 or the emergency server (not shown) sends the measurement information data received from the cradle 430 to a medical center 312 and receives diagnosis information analyzed by the medical center 312. Also, if necessary, the server 310 or the emergency server (not shown) can send the measurement information data to a communication terminal (not shown). Herein, the communication terminal may be any mobile communication terminal, such as a cellular phone, PDA, etc., or a personal computer. For example, according to the present invention, in a case where a person using the portable measurement unit 410 and the cradle 430 is a patient and a person receiving the measurement information data is a healthcare provider, if the patient couples the portable measurement unit 410 with the cradle 430 after measuring his or her human body by using the portable measurement unit 410, a measurement information data of the patient is transferred to a communication terminal (not shown) of the healthcare provider through a communication network, so that the healthcare provider inspects a health state of the patient.

If necessary, a clock (not shown) provided in the cradle 430 may be synchronized with a clock in the server 310. The reason is for preventing communication performance from degrading resulting from the clock error. That is, the communication performance may be degraded because the clock module, which is set to access to the server 310 at a first time, may try to access top the server 310 at a second time, which is different from the first time, due to the clock error. Also, a manual data transmission mode may be added. This function allows a user to transfer data specifically when the user wants to transmit data.

In case of emergency, an emergency signal is transferred through a procedure other than the general data transfer procedure described above. Firstly, the portable measurement unit 410 determines whether or not the biological measurement data exists within a range of the predetermined value by analyzing the biological measurement data, so that the portable measurement unit 410 determines whether or not an emergency occurs. Also, if a user notes that an emergency occurs, the user reports occurrence of the emergency by pressing an emergency button on the portable measurement unit 410. In case of such an emergency, the portable measurement unit 410 directly transfers a signal wirelessly to the cradle 430, the server 310, or the emergency server (not shown).

Signal transmission from the portable measurement unit 410 to the cradle 430 is achieved in such a manner that the portable measurement unit 410 first transfers an emergency signal to the cradle 430, the cradle 430 responds to the portable measurement unit 410 with a confirmation signal, and the portable measurement unit 410 transfers a re-confirmation signal to the cradle 430. If the portable measurement unit 410 does not receive the confirmation signal from the cradle 430, the portable measurement unit 410 continuously transfers the emergency signal to the cradle 430 by a predetermined time interval. If the cradle 430 does not receive the re-confirmation signal from the portable measurement unit 410, the cradle 430 transmits the confirmation signal to the portable measurement unit 410 again by a predetermined time interval. If the cradle 430 receives the re-confirmation signal, the cradle transmits an emergency signal to the server 310 or the emergency server (not shown). If the cradle 430 does not receive the re-confirmation signal, the cradle 430 determines that an emergency occurs and transmits the emergency signal to the server 310 or the emergency server (not shown). Herein, data transferred to the portable measurement unit 410, the cradle 430, the server 310 or the emergency server (not shown), the medical center or the communication terminal together with the emergency signal basically include an ID of the portable measurement unit 410 or/and an ID of the cradle 430. Also, the data additionally include a flag for indicating an emergency situation, a situation occurrence time, and related measurement data.

The cradle 430 can transfer the emergency signal to the server 310 or the emergency server (not shown) in a method similar to a data download procedure described above. Also, the cradle 430 can transfer the emergency signal to the server 310 or the emergency server (not shown) through different communication methods based on an emergency. For example, in the case of a general cable telephone communication employing a common channel signaling system, the cradle 430 can notify the server 310 or the emergency server (not shown) of occurrence of an emergency by combining caller ID transmission with a simple call connection to the server 310 or the emergency server. Also, in the case of a portable telephone communication, the cradle 430 can use a short message service in order to notify occurrence of an emergency. In addition, in the case of a cable telephone providing the SMS, the cradle 430 can use the SMS.

While this invention has been described in connection with what is presently considered to be the most practical and preferred embodiment, it is to be understood that the invention is not limited to the disclosed embodiment and the drawings, but, on the contrary, it is intended to cover various modifications and variations within the spirit and scope of the appended claims.

### INDUSTRIAL APPLICATION

Although a conventional tele-healthcare system is considered convenient because it can relieves a patient from going to a hospital himself/herself, it is very expensive as well as the usage thereof is very complex. However, the on-line healthcare system according to a preferred embodiment of the present invention does not require any additional personal computer in order to provide tele-healthcare management service, so that the on-line healthcare system is very economical. Also, on-line healthcare system according to a preferred embodiment of the present invention does not require an additional operation procedure, so that it is possible to achieve an easy-to-use on-line healthcare system.

In other words, since a portable measurement unit applied to an on-line healthcare system according to a preferred embodiment of the present invention is used in a method similar to or identical to a usage method of a general portable measurement unit, a special training for using the portable measurement unit is not required. Also, since data are transferred in a simple method, once the portable measurement unit is coupled with a cradle, data are automatically transferred through a program included in the portable measurement unit. Accordingly, usage of the portable measurement unit is very simple. Furthermore, since the cradle employs a simple communication method, the cradle has a few factors increasing a price thereof and does not require an additional accessory equipment. Therefore, the cradle is economical.

Also, the on-line healthcare system according to a preferred embodiment of the present invention can provide various services when the on-line healthcare system is connected to wireless communication. That is, the on-line healthcare system is connected to wireless communication in case of an emergency and is connected to wired communication ordinarily, so that the on-line healthcare system has a flexibility of providing different services according to various situations.

1. An on-line healthcare system by using a domestic medical device, the on-line healthcare system comprising:
a portable measurement unit for performing a biological measurement for diagnosing a user's health and converting measured data so as to generate biological measurement information data and/or measurement information data including the biological measurement data; and
a cradle connected to the portable measurement unit so as to automatically transmit/receive the measurement information data to/from the portable measurement unit by means of a program stored therein.

2. The on-line healthcare system as claimed in claim 1, further comprising a server connected to a communication network and including a database for storing measurement information data, the measurement information data being classified by collecting and analyzing the measurement information data transferred from the cradle.

3. The on-line healthcare system as claimed in claim 2, further comprising an emergency server having an emergency address capable of providing highly-reliable communication if an analysis of the biological measurement data results in an emergency situation.

4. The on-line healthcare system as claimed in claim 2, further comprising a medical center allowing a medical specialist to transfer diagnosis information about the measurement information data to the server or the emergency server by using the measurement information data received from the server or the emergency server.

5. The on-line healthcare system as claimed in claim 2, wherein the cradle automatically transmits or receives the measurement information data and the diagnosis information to or from the server by a predetermined time interval.

6. The on-line healthcare system as claimed in claim 2, wherein the cradle automatically transmits or receives the measurement information data and the diagnosis information to or from the server immediately after the portable measurement unit is coupled with the cradle.

7-8. (canceled)

9. The on-line healthcare system as claimed in claim 2, wherein the cradle makes communication with the server or the emergency server by using dual tone multi-frequency (DTMF).

10. (canceled)

11. The on-line healthcare system as claimed in claim 1, wherein the measurement information data includes at least a part or all of the biological measurement data, a measurement time of the biological measurement data, an ID of the portable measurement unit, and an ID of a user.

12-17. (canceled)

18. The on-line healthcare system as claimed in claim 1, wherein the cradle includes a second connection unit connected to the portable measurement unit or the server and/or a second central processing unit for processing, analyzing, or storing data.

19. The on-line healthcare system as claimed in claim 18, wherein the connection unit includes a second communication module for transmitting/receiving information using a second communication port of the cradle or the second communication port and wired/wireless communication.

20. The on-line healthcare system as claimed in claim 18, wherein the data includes at least one selected from the group consisting of the measurement information data, environment data, indication data for indicating whether or not new data exist, range indication data for indicating a range of the new data, and error data.

21. The on-line healthcare system as claimed in claim 20, wherein the environment data includes an address of the server and time for transferring the measurement information data.

22. The on-line healthcare system as claimed in claim 21, wherein the environment data further includes an emergency address of the server or an address of an emergency server for transferring the measurement information data if an analysis result of the biological measurement data measured by the portable measurement unit determines that an emergency occurs.

23. The on-line healthcare system as claimed in claim 20, wherein the environment data is remotely established and modified through information transferred from the server.

24-25. (canceled)

26. The on-line healthcare system as claimed in claim 13, wherein the first communication port and the second communication port have concavo-convex electrodes attached thereto, so that the portable measurement unit is coupled with the cradle.

27-29. (canceled)

30. The on-line healthcare system as claimed in claim 1, wherein the measurement information data temporarily stored in the portable measurement unit are delivered to the cradle when the portable measurement unit is coupled with the cradle.

31-33. (canceled)

34. An on-line healthcare method by using a domestic medical device including a portable measurement unit having a measurement part, a signal processing part, and a first communication module of the portable measurement unit, and a cradle having a program included therein and a second communication port of the cradle, the on-line healthcare method comprising the steps of:
(a) allowing the cradle to perform biological measurement for diagnosing health of a user;
(b) allowing the signal processing module to convert a result of the biological measurement into biological measurement data;
(c) determining whether or not an emergency occurs according to an analysis result of the biological measurement data measured by the portable measurement unit;
(d) transferring the measurement information data including a part of the biological measurement data to the cradle by using the second communication module of the cradle, the first communication module of the portable measurement unit, and the program included in the cradle, the cradle being automatically operated when the portable measuring unit makes contact with the cradle, if step (c) determines that no emergency occurs; and
(e) transferring the measurement information data received by the cradle to the server by using the program included in the cradle and the second communication module of the cradle.

35. The on-line healthcare method as claimed in claim 34, further comprising the step of (f) transferring the measurement information data received by the server to a medical center or a communication terminal.

36. The on-line healthcare method as claimed in claim 34, further comprising the steps of:
(d1) transferring an emergency signal to the cradle by wireless method by using the first communication module of the portable measurement unit, the second communication module of the cradle, the program stored in the cradle, the cradle being automatically operated when the portable measurement unit is contacted with the cradle, if step (c) determines that an emergency occurs; and
(d2) transferring the emergency signal received by the cradle to the server or an emergency server through the second communication module of the cradle.

37. The on-line healthcare method as claimed in claim 34, further comprising the step of (d3) wirelessly transferring an emergency signal to the server or an emergency server through the first communication module of the portable measurement unit if step (c) determines that an emergency occurs.

38. The on-line healthcare method as claimed in claim 36, further comprising the step of (d4) transferring the emergency signal received by the server or the emergency server to a medical center or a communication terminal.

39. The on-line healthcare method as claimed in claim 38, further comprising the steps of:
(g1) allowing the medical center to transfer diagnosis information to the server or the emergency server; and (g2) transferring the diagnosis information received by the server or the emergency server to the portable measurement unit.

40-42. (canceled)

43. The on-line healthcare method as claimed in claim 36, wherein the emergency signal is automatically transmitted by confirming a position of the portable measurement unit through a caller identification if the portable measurement unit or the cradle tries to perform call-connection to an emergency address of the server or an address of the emergency server.

44. (canceled)

45. The on-line healthcare method as claimed in claim 34, further comprising the steps of:
(g) allowing the medical center to transmit diagnosis information to the server or the emergency server;
(h) transferring the diagnosis information received by the server or the emergency server to the cradle; and
(i) transferring the diagnosis information received by the cradle to the portable measurement unit.

46. The on-line healthcare method as claimed in claim 36, wherein the cradle makes communication with the server or the emergency server on a basis of dual tone multi-frequency (DTMF).

47-51. (canceled)

52. The on-line healthcare method as claimed in claim 34, wherein the cradle includes a second connection unit connected to the portable measurement unit or the server and/or a second central processing unit for processing, analyzing, or storing data.

53. The on-line healthcare method as claimed in claim 52, wherein the data includes at least one selected from the group consisting of the measurement information data, environment data, indication data for indicating whether or not new data exist, range indication data for indicating a range of the new data, and error data.

54. The on-line healthcare method as claimed in claim 53, wherein the environment data includes a general address of the server and time for transferring the measurement information data.

55. The on-line healthcare method as claimed in claim 54, wherein the environment data further includes an emergency address of the server or an address of an emergency server for transferring the measurement information data if an analysis result of the biological measurement data measured by the portable measurement unit determines that an emergency occurs.

56. The on-line healthcare method as claimed in claim 53, wherein the environment data can be remotely established and modified through information transferred from the server.

57. (canceled)

58. The on-line healthcare method as claimed in claim 34, wherein the measurement information data temporarily stored in the portable measurement unit are delivered to the cradle when the portable measurement unit is coupled with the cradle.

59. The on-line healthcare method as claimed in claim 34, wherein when the portable measurement unit is coupled with the cradle, the program included in the cradle includes a program of automatically transmitting/receiving the measurement information data and a program of automatically trying connection of the server and the cradle at time predetermined by the program included in the cradle or right after the portable measurement unit is contacted with the cradle.

60-61. (canceled)

62. An on-line healthcare method by using a domestic medical device including a portable measurement unit having a measurement part, a signal processing part and a first communication module of the portable measurement unit, and a cradle having a program included therein and a second communication port of the cradle, the on-line healthcare method comprising the steps of:
(a) allowing the cradle to perform biological measurement for diagnosing health of a user;
(b) allowing the signal processing module to convert a result of the biological measurement into biological measurement data;
(c) transferring the measurement information data including a portion of the biological measurement data to the cradle by using the first communication module of the portable measurement unit, the second communication module of the cradle, and the program included in the cradle, the cradle being automatically operated when the portable measuring unit is contacted with the cradle; and
(d) transferring the measurement information data received by the cradle to the server by using the program included in the cradle and the second communication module of the cradle.

63. The on-line healthcare method as claimed in claim 62, further comprising a step of (e) transferring the measurement information data received by the server to a medical center or a communication terminal.

64. The on-line healthcare method as claimed in claim 63, further comprising the steps of:
(f) allowing the medical center to transmit diagnosis information to the server;
(g) transferring the diagnosis information to the cradle; and
(h) transferring the diagnosis information received by the mounting server to the portable measurement unit.

65. The on-line healthcare system as claimed in claim 3, further comprising a medical center allowing a medical specialist to transfer diagnosis information about the measurement information data to the server or the emergency server by using the measurement information data received from the server or the emergency server.

66. The on-line healthcare system as claimed in claim 3, wherein the cradle automatically transmits or receives the measurement information data and the diagnosis information to or from the server by a predetermined time interval.

67. The on-line healthcare system as claimed in claim 3, wherein the cradle automatically transmits or receives the measurement information data and the diagnosis information to or from the server immediately after the portable measurement unit is coupled with the cradle.

68. The on-line healthcare system as claimed in claim 3, wherein the cradle makes communication with the server or the emergency server by using dual tone multi-frequency (DTMF).

69. The on-line healthcare system as claimed in claim 19, wherein the first communication port and the second communication port have concavo-convex electrodes attached thereto, so that the portable measurement unit is coupled with the cradle.

70. The on-line healthcare method as claimed in claim 37, further comprising the step of (d4) transferring the emergency signal received by the server or the emergency server to a medical center or a communication terminal.

71. The on-line healthcare method as claimed in claim 37, wherein the emergency signal is automatically transmitted by confirming a position of the portable measurement unit through a caller identification if the portable measurement unit or the cradle tries to perform call-connection to an emergency address of the server or an address of the emergency server.

72. The on-line healthcare method as claimed in claim 37, wherein the cradle makes communication with the server or the emergency server on a basis of dual tone multi-frequency (DTMF).

73. The on-line healthcare method as claimed in claim 45, wherein the cradle makes communication with the server or the emergency server on a basis of dual tone multi-frequency (DTMF).
